(19) United States
(12) Patent Application Publication
(10) Pub. No.: US 2025/0248767 A1
(43) Pub. Date: Aug. 7, 2025

(54) ULTRASOUND IMAGING METHOD, ULTRASOUND IMAGING SYSTEM, AND NON-TRANSITORY COMPUTER-READABLE MEDIUM

(71) Applicant: GE Precision Healthcare LLC, Waukesha, WI (US)

(72) Inventors: Ke Tao, Wuxi (CN); Yi Wang, Wuxi (CN); Xingti Liu, Wuxi (CN); Benfang Wu, Wuxi (CN); Marvin Ler, Singapore (SG)

(21) Appl. No.: 19/044,358

(22) Filed: Feb. 3, 2025

(30) Foreign Application Priority Data
Feb. 2, 2024 (CN) 202410153304.5

(51) Int. Cl. A61B 34/20; A61B 8/00; A61B 8/08

(52) U.S. Cl. CPC A61B 34/20; A61B 8/0841; A61B 8/463; A61B 8/54; A61B 2034/2063

(57) ABSTRACT

A method for guiding movement of a probe, so as to perform ultrasound imaging, comprising: using the probe to acquire a real-time ultrasound image related to a tissue to be imaged; determining a current intervention starting point, and determining a target intervention region and a non-intervention region in the ultrasound image; determining a current intervention path based on the target intervention region and the current intervention starting point, and determining a target intervention path based on the target intervention region and the non-intervention region; and, on the basis of the current intervention path and the target intervention path, generating and displaying movement guidance related to the probe, the movement guidance being configured to guide the probe to move such that the current intervention path and the target intervention path coincide.

### CROSS-REFERENCE TO RELATED APPLICATION

This application claim priority to Chinese Patent Application No. 202410153304.5, which was file on Feb. 2, 2024 at the Chinese Patent Office. The entire contents of the above-listed application are incorporated by reference herein in their entirety.

### TECHNICAL FIELD

The present invention relates to the field of ultrasound imaging and, in particular, to an ultrasound imaging method, an ultrasound imaging system, and a non-transitory computer-readable medium.

### BACKGROUND

Ultrasound imaging is a real-time non-invasive imaging technology. It utilizes a probe to transmit an ultrasound beam to a tissue to be imaged and receive an ultrasound echo signal. Processing the ultrasound echo signal can generate an ultrasound image related to the tissue to be imaged. In some application scenarios, ultrasound imaging can assist in determination of the position of a target intervention region (e.g., a lesion) in a surgical intervention (e.g., a biopsy or treatment) in real time, so as to assist doctors in planning an intervention path.

Ultrasound-assisted interventions are widely used in many scenarios. For example, superficial subcutaneous interventions, including interventions for thyroids, breasts, etc., or abdominal interventions, such as for the liver, etc., are widely assisted by ultrasound imaging. In particular, doctors can determine the location of target regions (e.g., lesions) as well as non-intervention regions (e.g., blood vessels, normal glandular tissues, nerves, etc.) from ultrasound images. As a result, doctors can plan and adjust intervention paths so that the intervention paths can reach the target regions while avoiding the non-intervention regions. Intervention path planning is typically performed for an interventional object (e.g., a needle).

### SUMMARY

The inventors have found that current means of intervention path adjustment have some drawbacks. One of the drawbacks is that a doctor often needs to adjust an intervention starting point (e.g., a puncture site on the skin) and an intervention angle of an interventional object depending on different individual circumstances. Such starting point and angle adjustment manner is not regular, and depends largely on the doctor's experience. A second drawback is that when the intervention starting point of the interventional object is adjusted, the intervention starting point may in some cases exceed the range of an ultrasound image, e.g. in order to avoid tissues that are not desired to be punctured (non-intervention regions), it may be necessary to set the intervention starting point and the intervention angle to be relatively angled, such that the intervention starting point is far away from a probe. As a result, the ultrasound image does not completely cover the entire intervention path, and the doctor needs to operate relying on experience at the beginning of an interventional procedure, rather than relying on ultrasound guidance. This causes the efficiency and accuracy of the ultrasound imaging assisted intervention to be low.

The aforementioned defects, deficiencies, and problems are solved herein, and these problems and solutions will be understood through reading and understanding the following description.

Some embodiments of the present application provide a method for guiding movement of a probe to perform ultrasound imaging, comprising: using the probe to acquire a real-time ultrasound image related to a tissue to be imaged; determining a current intervention starting point, and determining a target intervention region and a non-intervention region in the ultrasound image; determining a current intervention path based on the target intervention region and the current intervention starting point, and determining a target intervention path based on the target intervention region and the non-intervention region; and, on the basis of the current intervention path and the target intervention path, generating and displaying movement guidance related to the probe, the movement guidance being configured to guide the probe to move such that the current intervention path and the target intervention path coincide.

Some other embodiments of the present application provide an ultrasound imaging system, comprising: a probe, a processor, and a display apparatus. The probe transmits an ultrasound beam to a tissue to be imaged and receives an echo signal. The display apparatus receives a control of the processor and performs a display operation. The processor is configured to: use the probe to acquire a real-time ultrasound image related to a tissue to be imaged; determine a current intervention starting point, and determine a target intervention region and a non-intervention region in the ultrasound image; on the basis of the target intervention region and the target intervention region, determine a current intervention path, and on the basis of the target intervention region and the non-intervention region, determine a target intervention path; and, on the basis of the current intervention path and the target intervention path, generate and display movement guidance related to the probe, the movement guidance being configured to guide the probe to move such that the current intervention path and the target intervention path coincide.

Some embodiments of the present application also provide a non-transitory computer-readable medium, the non-transitory computer-readable medium having a computer program stored thereon, the computer program having at least one code segment, and the at least one code segment being executable by a machine so as to cause the machine to: use the probe to acquire a real-time ultrasound image related to a tissue to be imaged; determine a current intervention starting point, and determine a target intervention region and a non-intervention region in the ultrasound image; on the basis of the target intervention region and the target intervention region, determine a current intervention path, and on the basis of the target intervention region and the non-intervention region, determine a target intervention path; and, on the basis of the current intervention path and the target intervention path, generate and display movement guidance related to the probe, the movement guidance being configured to guide the probe to move such that the current intervention path and the target intervention path coincide.

It should be understood that the brief description above is provided to introduce, in a simplified form, concepts that will be further described in the detailed description. The brief description above is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### BRIEF DESCRIPTION OF THE DRAWINGS

The present application will be better understood by reading the following description of non-limiting embodiments with reference to the accompanying drawings, where:

FIG. 1 is a schematic diagram of an ultrasound imaging system according to some embodiments of the present application;

FIG. 2 is a flowchart of a method for guiding movement of a probe to perform ultrasound imaging according to some embodiments of the present application;

FIG. 3 is a schematic illustration of performing intervention path planning by means of adjusting an intervention starting point according to some embodiments of the present application;

FIG. 4 is a schematic diagram of intervention path planning in some embodiments of the present application;

FIG. 5 is a schematic diagram showing a probe guided to move after an intervention path is planned in some embodiments of the present application; and

FIG. 6 is a schematic diagram of ultrasound imaging during an intervention according to some embodiments of the present application.

### DETAILED DESCRIPTION

Specific implementations of the present invention will be described below. It should be noted that in the specific description of the implementations, it is impossible to describe all features of the actual implementations of the present invention in detail, for the sake of brief description. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless otherwise defined, the technical or scientific terms used in the claims and the description should be as they are usually understood by those possessing ordinary skill in the technical field to which they belong. “First”, “second”, and similar words used in the present invention and the claims do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

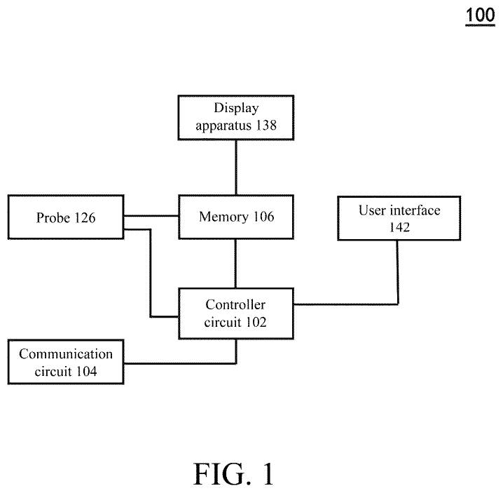

FIG. 1 shows a schematic block diagram of an embodiment of an ultrasound imaging system 100. The ultrasound imaging system 100 may include a controller circuit 102, a display apparatus 138, a user interface 142, a probe 126, and a memory 106, which can be operatively connected to a communication circuit 104.

The controller circuit 102 is configured to control operation of the ultrasound imaging system 100. The controller circuit 102 may include one or more processors. Optionally, the controller circuit 102 may include a central processing unit (CPU), one or more microprocessors, a graphics processing unit (GPU), or any other electronic component capable of processing inputted data according to a specific logic instruction. Optionally, the controller circuit 102 may include and/or represent one or more hardware circuits or circuit systems, and the hardware circuit or circuit system includes, is connected to, or includes and is connected to one or more processors, controllers, and/or other hardware logic-based apparatuses. Additionally or alternatively, the controller circuit 102 may execute an instruction stored on a tangible and non-transitory computer-readable medium (e.g., the memory 106).

The controller circuit 102 may be operatively connected to and/or control the communication circuit 104. The communication circuit 104 is configured to receive and/or transmit information along a bidirectional communication link with one or more alternate ultrasound imaging systems, remote servers, etc. The remote server may represent patient information, a machine learning algorithm, a remotely stored ultrasound image from a previous scan and/or diagnosis and treatment period of a patient, etc. The communication circuit 104 may represent hardware for transmitting and/or receiving data along a bidirectional communication link. The communication circuit 104 may include a transceiver, a receiver, etc., and an associated circuit system (e.g., an antenna) for communicating (e.g., transmitting and/or receiving) with the one or more alternate ultrasound imaging systems, remote servers, etc., by using a wired and/or wireless means. For example, protocol firmware for transmitting and/or receiving data along a bidirectional communication link may be stored in the memory 106 accessed by the controller circuit 102. The protocol firmware provides network protocol syntax to the controller circuit 102 so as to assemble a data packet, establish and/or segment data received along the bidirectional communication link, and so on.

The bidirectional communication link may be a wired (e.g., by means of a physical conductor) and/or wireless communication (e.g., utilizing a radio frequency (RF)) link for exchanging data (e.g., a data packet) between the one or more alternative ultrasound imaging systems, remote servers, etc. The bidirectional communication link may be based on a standard communication protocol, such as Ethernet, TCP/IP, Wi-Fi, 802.11, a customized communication protocol, Bluetooth, etc.

The controller circuit 102 is operatively connected to the display apparatus 138 and the user interface 142. The display apparatus 138 may include one or more liquid crystal display apparatuses (e.g., light emitting diode (LED) backlights), organic light emitting diode (OLED) display apparatuses, plasma display apparatuses, CRT display apparatuses, and the like. The display apparatus 138 may display patient information, one or more ultrasound images and/or videos, a graphical user interface, or a component received by the display apparatus 138 from the controller circuit 102, one or more 2D, 3D or 4D ultrasound image data sets from ultrasound data stored in the memory 106, or an anatomical measurement, diagnosis, processing information etc. currently acquired in real time.

The user interface 142 controls the operation of the controller circuit 102 and the ultrasound imaging system 100. The user interface 142 is configured to receive an input from a clinician and/or an operator of the ultrasound imaging system 100. The user interface 142 may include a keyboard, a mouse, a trackball, a touch pad, one or more physical buttons, and the like. Optionally, the display apparatus 138 may be a touch screen display apparatus that includes at least a part of the user interface 142. For example, a part of the user interface 142 may correspond to a graphical user interface (GUI) that is generated by the controller circuit 102 and is shown on the display apparatus 138. The touch screen display apparatus may detect the presence of a touch from the operator on the display apparatus 138, and may also identify the location of the touch relative to the surface area of the display apparatus 138. For example, a user may select, by touching or contacting the display apparatus 138, one or more user interface components of the user interface (GUI) shown on the display apparatus. User interface components may correspond to icons, text boxes, menu bars, etc., shown on the display apparatus 138. A clinician may select, control, and use a user interface assembly, interact with the same, and so on, so as to send an instruction to the controller circuit 102 to perform one or more operations described in the present application. For example, a touch may be applied using at least one among a hand, a glove, a stylus, and the like.

The memory 106 includes a parameter, an algorithm, one or more protocols of ultrasound examination, data values, and the like used by the controller circuit 102 to execute one or more operations described in the present application. The memory 106 may be a tangible and non-transitory computer-readable medium such as a flash memory, a RAM, a ROM, an EEPROM, etc. The memory 106 may include a set of learning algorithms (e.g., a convolutional neural network algorithm, a deep learning algorithm, a decision tree learning algorithm, etc.) configured to define an image analysis algorithm. During execution of the image analysis algorithm, the controller circuit 102 is configured to identify a section (or a view or an anatomical plane) of an anatomical structure of interest in an ultrasound image. Optionally, an image analysis algorithm may be received by means of the communication circuit 104 along one among bidirectional communication links, and stored in the memory 106.

The image analysis algorithm may be defined by one or more algorithms, so as to identify one or more anatomical features (e.g., a boundary, a thickness, a pixel value change, a valve, a cavity, a chamber, an edge or inner layer, a vessel structure, etc.) in an ultrasound image. The one or more anatomical features may represent a feature of pixels and/or voxels of the ultrasound image, such as a histogram of oriented gradients, a point feature, a covariance feature, a binary mode feature, and the like. For example, the image analysis algorithm may be defined by using prediction of object identification within the ultrasound image using one or more deep neural networks. During ultrasound imaging assisting an intervention, the image analysis algorithm described above is also suitable for identifying an interventional object (e.g., a needle) in the ultrasound image. An exemplary description of an implementation of the image analysis algorithm is described below.

The image analysis algorithm may correspond to an artificial neural network formed by the controller circuit 102 and/or the remote server. The image analysis algorithm may be divided into two or more layers, such as an input layer for receiving an input image, an output layer for outputting an output image, and/or one or more intermediate layers. Layers of a neural network represent different groups or sets of artificial neurons, and may represent different functions that are executed by the controller circuit 102 on an input image (e.g., an ultrasound image acquired and/or generated by the ultrasound imaging system 100) so as to identify features contained in the input image. An artificial neuron in a layer of the neural network may examine an individual pixel in the input image. The artificial neurons use different weights in a function applied to the input image, so as to attempt to identify an object in the input image. The neural network produces an output result by assigning or associating different pixels in the output image with different anatomical features, on the basis of analysis of pixel characteristics.

In some examples, the image analysis algorithm is defined by a plurality of training images; the plurality of training images may be grouped into ultrasound images of different tissues to be imaged, and trained to identify different anatomical features in ultrasound images in the different tissues to be imaged. According to actual need, the anatomical features may be classified into different types, e.g., a lesion such as a tumor, or a normal anatomical feature such as a blood vessel, a gland, or a ganglion point. Additionally or alternatively, the image analysis algorithm may be defined by the controller circuit on the basis of a classification model. The classification model may correspond to a machine learning algorithm based on a classifier (e.g., a random forest classifier, principal component analysis, etc.) configured to identify and/or assign an anatomical feature to multiple types or categories on the basis of overall shape, spatial position of the anatomical structure, signal intensity, etc.

In some other embodiments, the image analysis algorithm of the controller circuit 102 may also be any other means in the art. For example, the image analysis algorithm may be an algorithm that identifies a boundary of a structure of interest in an image on the basis of changes in an image grayscale value. For example, a certain grayscale value threshold may be set, and neighboring pixel points in the image are compared by using the threshold. If the grayscale value of a neighboring pixel point is greater than the above-described grayscale value threshold, it is determined that said position is a position to be identified, e.g., may be a boundary of a structure of interest. The structure of interest may be a lesion (e.g., a tumor). In addition, it may also be determined that the position is the position at which an interventional object (e.g., a needle) is located, thereby identifying the interventional object. In addition, it should be noted that the example of image identification performed by the controller circuit 102 is described in the foregoing embodiment of the present application, but identification means are not limited thereto, and are not further enumerated herein.

In an application scenario for an intervention, the described image analysis algorithm can give the ultrasound imaging system 100 functionality to assist the intervention. For example, the image analysis algorithm enables the ultrasound imaging system 100 to identify a target intervention region (e.g., a lesion), as well as a non-intervention region (e.g., a blood vessel, a gland, a ganglion point, etc.) in an ultrasound image. The image analysis algorithm may also be used to identify an interventional object in the ultrasound image. Thus, doctors may be provided with key information needed for interventional imaging. Specific assistance means will be described in detail below.

With further reference to FIG. 1, the ultrasound imaging system 100 may include the probe 126. The probe 126 has elements such as an ultrasound transducer, a transmitter, a transmit beam former, a detector/SAP electronics, etc., (not shown). The detector/SAP electronics may be used to control the switching of the transducer elements. The detector/SAP electronics may also be used to group the transducer elements into one or more sub-holes. Configurations of the probe 126 will also be described below exemplarily.

The probe 126 may be configured to acquire ultrasound data or information from tissue to be imaged of a patient. The probe 126 is communicatively connected to the controller circuit by means of the transmitter. The transmitter transmits a signal to the transmit beam former on the basis of acquisition settings received by the controller circuit 102. The acquisition settings may define the amplitude, pulse width, frequency, gain setting, scanning angle, power, time gain compensation (TGC), resolution, and the like of the ultrasound pulses emitted by the ultrasound transducer. The ultrasound transducer emits a pulsed ultrasound signal into a patient (e.g., the body). The acquisition settings may be defined by a user operating the user interface 142. The signal transmitted by the transmitter, in turn, drives the ultrasound transducer.

The ultrasound transducer transmits the pulsed ultrasound signal to a body (e.g., a patient) or a volume that corresponds to an acquisition setting along one or more scanning planes. The ultrasound signal may include, for example, one or more reference pulses, one or more push pulses (e.g., shear waves), and/or one or more pulsed wave Doppler pulses. At least a portion of the pulsed ultrasound signal is backscattered from the tissue to be imaged (e.g., the superficial skin, the thyroid, the breast, the prostate, etc. to be intervened) to generate an echo. Depending on the depth or movement, the echo is delayed in time and/or frequency, and received by the ultrasound transducer. The ultrasound signal may be used for imaging, for producing and/or tracking a shear wave, for measuring changes in location or velocity within the anatomical structure and a compressive displacement difference (e.g., strain) of tissue, and/or for treatment and other applications. For example, the probe 126 may deliver low energy pulses during imaging and tracking, deliver medium and high energy pulses to produce shear waves, and deliver high energy pulses during treatment.

The ultrasound transducer converts a received echo signal into an electrical signal that can be received by the receiver. The receiver may include one or more amplifiers, analog/digital converters (ADCs), and the like. The receiver may be configured to amplify the received echo signal after appropriate gain compensation, and convert these analog signals received from each transducer element into a digitized signal that is temporally uniformly sampled. The digitized signals representing the received echoes are temporarily stored in the memory 106. The digitized signals correspond to the backscattered waves received by each transducer element at different times. After being digitized, the signal may still retain the amplitude, frequency, and phase information of the backscattered wave.

Optionally, the controller circuit 102 may retrieve the digitized signals stored in the memory 106 for use in a beam former processor. For example, the controller circuit 102 may convert the digitized signal into a baseband signal or compress the digitized signal.

In some embodiments, the controller circuit 102 may further include a beam forming processor. The beam forming processor may include one or more processors. If desired, the beam forming processor may include a central processing unit (CPU), one or more microprocessors, or any other electronic component capable of processing the input data according to specific logic instructions. Additionally or alternatively, the beam forming processor may execute instructions stored on a tangible and non-transitory computer-readable medium (e.g., the memory 106) to perform beam forming computation using any suitable beam forming method, such as adaptive beam forming, synthetic emission focusing, aberration correction, synthetic aperture, clutter suppression, and/or adaptive noise control, etc.

In some embodiments, the controller circuit 102 may further include a radio frequency (RF) processor. The beam forming processor executes beam forming on the digitized signals of the transducer elements, and outputs an RF signal. The RF signal is then provided to the RF processor for processing the RF signal. The RF processor may include one or more processors. If desired, the RF processor may include a central processing unit (CPU), one or more microprocessors, or any other electronic component capable of processing the inputted data according to specific logic instructions. Additionally or alternatively, the RF processor may execute instructions stored on a tangible and non-transitory computer-readable medium (e.g., the memory 106). Optionally, the RF processor may be integrated with and/or be part of the controller circuit 102. For example, operations described as being executed by the RF processor may be configured to be executed by the controller circuit 102.

The RF processor may generate, for a plurality of scanning planes or different scanning modes, different ultrasound image data types and/or modes, e.g., B-mode, color Doppler (e.g., color blood flow, velocity/power/variance), tissue Doppler (velocity), and Doppler energy, on the basis of a predetermined setting of a first model. For example, the RF processor may generate tissue Doppler data for multiple scanning planes. The RF processor acquires the information (e.g., I/Q, B-mode, color Doppler, tissue Doppler, and Doppler energy information) related to a plurality of data pieces, and stores the data information in the memory 106, where the data information may include time stamp and orientation/rotation information.

Optionally, the RF processor may include a composite demodulator (not shown) for demodulating the RF signal to generate an IQ data pair representing an echo signal. The RF or IQ signal data may be provided directly to the memory 106 so as to be stored (e.g., stored temporarily). If desired, an output of the beam forming processor may be delivered directly to the controller circuit 102.

The controller circuit 102 may be configured to process the acquired ultrasound data (e.g., RF signal data or IQ data pairs), and prepare and/or generate frames of ultrasound image data representing an anatomical structure of interest so as to display the same on the display apparatus 138. The acquired ultrasound data may be processed by the controller circuit 102 in real time during a scanning or treatment process of ultrasound examination when echo signals are received. Additionally or alternatively, the ultrasound data may be temporarily stored in the memory 106 during a scanning process, and processed in a less real-time manner in a live or off-line operation.

The memory 106 may be used to store processed frames of acquired ultrasound data that are not scheduled to be immediately displayed, or may be used to store post-processed images (e.g., shear wave images and strain images), firmware or software corresponding to, for example, a graphical user interface, one or more default image display settings, programmed instructions, and the like. The memory 106 may store ultrasound images and can be based on a user input, e.g., a user selection received at the user interface 142.

In some examples, the described ultrasound image displayed on the display apparatus 138 may be a shear wave image, a 3D image or a 4D image. In some typical use scenarios, for example, in interventional imaging, the described image may be a 2D ultrasound image, 2D imaging being performed by means of selecting an appropriate probe 126. 2D ultrasound images are able to provide doctors with high-frame-rate and high-resolution ultrasound images, thereby facilitating performance of a real-time interventional surgery.

As described above in the present application, the inventors have found that, during an ultrasound imaging-assisted intervention, adjustment of the position of an interventional object faces the technical problems of being highly dependent on operator experience, and being prone to cause intervention failure. In response to the above problems, improvements are provided in one or more embodiments of the present application.

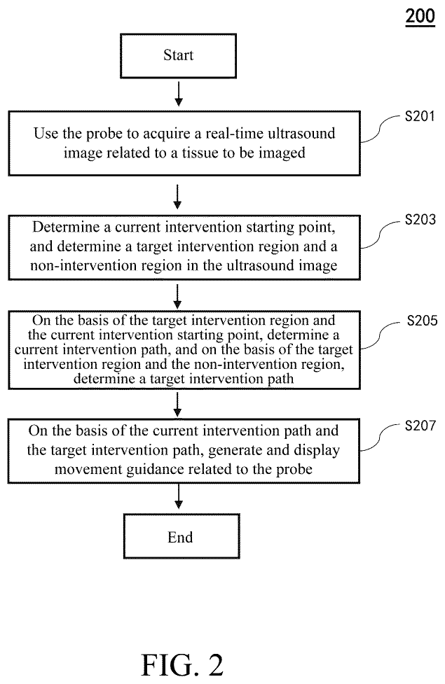

Reference is made to FIG. 2 which shows a flowchart of a method 200 for guiding a movement of a probe to perform ultrasound imaging according to some embodiments of the present application. The method 200 may include:

Step 201: using the probe to acquire a real-time ultrasound image related to a tissue to be imaged. This procedure may be performed by a processor; in particular, the processor may control the probe to transmit an ultrasound beam to a tissue to be imaged and receive an echo signal using the probe, then generate a real-time ultrasound image related to the tissue to be imaged by means of processing the echo signal.

Step 203: determining a current intervention starting point, and determining a target intervention region and a non-intervention region in the ultrasound image. The intervention starting point may be understood as a point of contact of an interventional object with a surface of the tissue to be imaged. For example, when a breast, a thyroid gland, a liver, or the like is punctured, the intervention starting point is a puncture point on the skin surface. Correspondingly, the current intervention starting point is also the puncture point (contact point) of the interventional object on the surface of the tissue to be imaged in a currently planned intervention path. The target intervention region is a target tissue on which a surgical intervention is performed using the interventional object, e.g., a lesion. In addition, the doctor may perform different types of operations, such as sampling or administering drugs, using the interventional object. It should be understood, however, that the above explanation is only intended to more clearly describe the assistance function of the ultrasound imaging technique of the present application, and that no interventional operation is included in the embodiments of the present application. The non-intervention region may be understood as a region that is not desired to be contacted by the interventional object, i.e., a region that needs to be avoided in an intervention. These regions are often important for the maintenance of health, e.g., blood vessels, normal glands, ganglion points, etc., which will not be exhaustively listed.

Step 205: determining a current intervention path on the basis of the target intervention region and the current intervention starting point, and determining a target intervention path on the basis of the target intervention region and the non-intervention region. For example, the processor, after determining the target intervention region and the current intervention starting point, may determine the current intervention path by means of a line connecting the two points. The target intervention path may then be an intervention path that can reach the target intervention region and does not pass through the non-intervention region.

Step 207: on the basis of the current intervention path and the target intervention path, generating and displaying movement guidance related to the probe, the movement guidance being configured to guide the probe to move such that the current intervention path and the target intervention path coincide. By means of determining a difference between the current intervention path and the target intervention path, the processor can generate guidance to move the probe and eliminate the difference.

In the above means of the present application, the movement guidance for the probe can be generated after the current intervention path and the target intervention path are determined and a difference between the two is determined, so as to help a user to quickly and accurately move the probe to an appropriate position for imaging assistance for the intervention. In an existing solution for adjusting a puncture site and an angle of the interventional object, a visual field of the interventional object may be partially lost in the ultrasound image. Moreover, the adjustment of the intervention path (site, angle, etc.) is difficult to follow regularly. In the solution of the present application, by means of adjusting the position of the probe, it can be effectively ensured that the field-of-view range of the ultrasound image is also adjusted during adjustment of the intervention path, so that better image guidance can be provided for the user, and the problem of an intervention starting point and part of the intervention path not being coverable by the ultrasound image can be avoided.

The movement guidance may be specifically set by various means. Unless otherwise specified, any symbol, text, or the like that is displayed on the display apparatus and can visually guide the movement of the probe may be the movement guidance. For example, in one example, the movement guidance may be an arrow. The direction of the arrow represents a movement direction of the probe. In a preferred example, the length of the arrow may also represent the amount of translation that the probe should have. In another example, the movement guidance may also be a text prompt related to a movement direction, a movement distance, etc. Furthermore, in one example, the movement guidance may be both the target intervention path and the current intervention path, and the user may move the probe himself by means of the difference between the two paths. In a further example, the movement guidance may also be the current intervention starting point and the target intervention starting point, a difference between the two starting points also serving the purpose of guiding the movement of the probe. It is to be understood that any of the examples described above may also be freely combined.

It is to be understood that the current intervention starting point may be manually set or may be set by the system as a default. In some embodiments, the current intervention starting point is set to be close to one side edge of the probe and located at an upper edge of the ultrasound image. The current intervention starting point is set at one side edge of the probe, which can facilitate a subsequent intervention operation and conform more to the user's habit. On the one hand, this can ensure that the position of the intervention starting point relative to the probe is relatively fixed, which reduces the dependence on operation technique and habit. On the other hand, being close to one side edge of the probe can also ensure that the intervention starting point remains within the field of view of the ultrasound image, and is not lost. In an actual ultrasound scan, the upper edge of the ultrasound image is the portion of the probe that is in contact with the tissue to be imaged (e.g., skin). Accordingly, the intervention starting point is also located at the upper edge of the ultrasound image.

Further, in some examples, the current intervention starting point moves in real time with the movement of the probe, and the relative positions of the current intervention starting point and the probe remain unchanged during the movement. In other words, the relative positions of the intervention starting point and the probe are fixed; for example, no matter how the probe moves, the intervention starting point is set to be close to one side edge of the probe, thereby achieving that the relative positions of the two remain unchanged. When the probe is moved, the tissue being imaged will change (e.g., translate). Once the change is reflected on the ultrasound image, ultrasound image is also translated, and the current intervention starting point is translated along with it. Although the distance of translation may be detected by a position sensor, in a preferred example, no additional device is required. In one example, a distance that the probe is moved can be determined just by means of the amount of translation of the image, and accordingly, a distance that the intervention starting point is moved can also be known. Alternatively, when the field of view of the probe is fixed, the position of the upper edge of the ultrasound image corresponding to one side edge of the probe is also fixed, and no matter how the probe moves, the position corresponding to one side edge of the probe does not change.

It should be noted that the intervention starting points (including the current intervention starting point and the target intervention starting point) in the example of the present application are reference points used to calculate, for example, the current intervention path, the target intervention path, and the probe movement guidance, when the processor executes an algorithm. The aforementioned intervention starting points are not directly related to a surgical intervention.

In the example of the present application, determination of the target intervention region and the non-intervention region in the ultrasound image may utilize any image recognition technique. For example, any technique such as machine learning, deep learning, or the like described in the above embodiments of the present application may be utilized. In one example, a deep learning technique may be utilized for identification. A plurality of deep neural networks may be trained for the target intervention region and the non-intervention region, respectively, and stored in the ultrasound imaging system to be called by the processor. The identification means may also another type, which types will not be further enumerated.

The embodiments of the present application will be described more clearly below with reference to an ultrasound image.

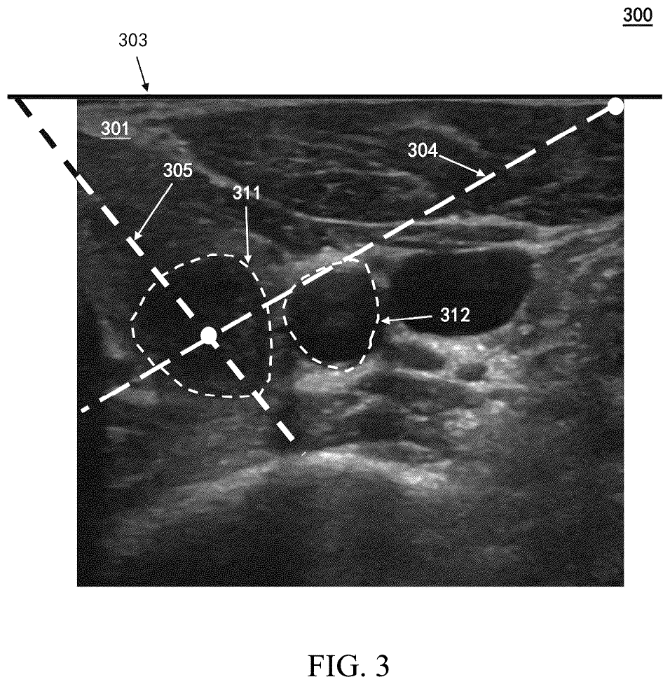

Reference is made first to FIG. 3, which shows a schematic diagram 300 of performing intervention path planning by means of adjusting intervention starting points. It will be appreciated that in a conventional ultrasound scan, an ultrasound image is displayed on a display, and for case of illustration, the ultrasound image is positioned directly below the skin for case of understanding in the present application.

As shown in FIG. 3, an ultrasound image 301 is obtained by processing an ultrasound signal transmitted to and received from a tissue to be imaged under skin 303 by a probe. A target intervention region 311 and a non-intervention region 312 may be determined by means of image recognition or the like. The target intervention region 311 may be a lesion to be intervened, and the non-intervention region 312 may be a site that is inappropriate to puncture, such as a blood vessel. In a current ultrasound image, although an intervention path 304 can be entirely covered by the ultrasound image, the intervention path passes through the non-intervention region 312, so the doctor cannot select the path. Therefore, it is necessary to adjust the intervention path 304, e.g., select another intervention path 305. It is not difficult to see that the intervention path 305 can avoid the non-intervention region 312 in the ultrasound image 301; however, a portion of the intervention path 305 is outside the image range of the ultrasound image 301. In a conventional operation, the doctor needs to rely on experience to perform a blind operation over a distance until an interventional object appears in the field of view. Drawbacks to this approach include excessive dependence on the operation technique and experience of the doctor, and an inability to provide comprehensive ultrasound imaging guidance, as well as whether there are non-intervention regions outside the image.

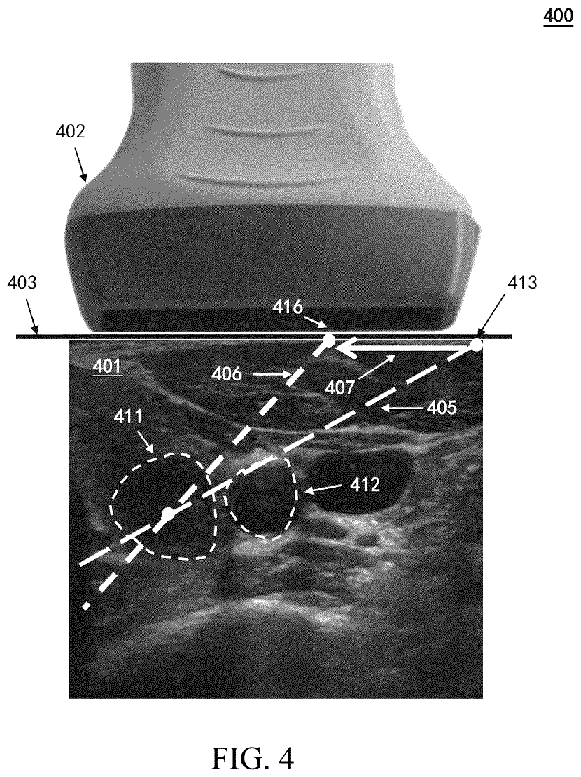

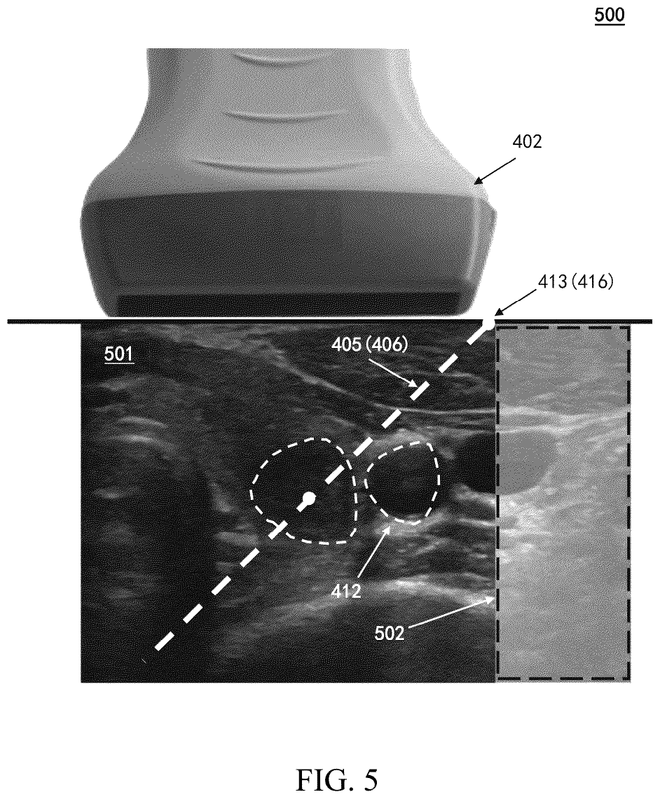

In the embodiments of the present application, path planning is not for the interventional object, but rather for the probe. Also, in a preferred example, the intervention starting point moves with the movement of the probe and always maintains a fixed relative position to the probe. In this way, it is ensured that, on the one hand, the field of view of the ultrasound image can change with a change of path planning (since the field of view is determined by the probe). On the other hand, it can also be ensured that the interventional object is not lost from the field of view, and the position of the intervention starting point is more fixed when the intervention starting point of the interventional object and the probe remain relatively fixed. The embodiments of the present application will be described in more detail below with reference to FIGS. 4 and 5, wherein FIG. 4 is a schematic diagram 400 of intervention path planning in some embodiments of the present application, and FIG. 5 is a schematic diagram 500 showing a probe which has been guided to move after an intervention path is planned in some embodiments of the present application.

Reference is made to FIG. 4 first. Similar to what is shown in FIG. 3, an ultrasound image 401 is obtained by processing an ultrasound signal transmitted to and received from an tissue to be imaged under skin 403 by a probe 402. A target intervention region 411 and a non-intervention region 412 may be determined by means of image recognition or the like (e.g., means described in any of the embodiments above). Further, in the embodiments of FIG. 4, a determined current intervention starting point 413 is also included. The current intervention starting point 413 is set to be close to one side edge of the probe 402 and located at an upper edge of the ultrasound image 401. It will be appreciated that the manner in which the current intervention starting point 413 is determined may be predetermined, e.g., preset in an ultrasound imaging system. “Close to” means pressing close to one side edge of the probe 402, and allowing a certain distance (e.g., a distance of 0.5-3 cm) so as not to interfere with operation of an interventional object by a doctor. Additionally, and optionally, the position of the current intervention starting point 413 may also allow a user to make an adjustment. For example, the position of the current intervention starting point 413 may also be adjusted by means of presetting in the ultrasound imaging system, or by means of operating a display apparatus (e.g., a touch screen), which will not be elaborated on here.

Further, after the current intervention starting point 413 and the target intervention region 411 are determined, the current intervention path 405 may be determined. In one embodiment, the current intervention path 405 may be a line connecting a point (e.g., a center point) in the target intervention region 411 to the current intervention starting point 413.

After the target intervention region 411 and the non-intervention region 412 are determined, a target intervention path 406 may also be determined. In one embodiment, the target intervention path 406 may be a line that passes through a point (e.g., a center point) of the target intervention region 411 and does not pass through the non-intervention region 412. In this way, it is possible to satisfy the purpose that the target intervention region can be reached and the non-intervention region is not punctured. Furthermore, it is not difficult to understand that there may be more than one target intervention path 406 that satisfies the above conditions. In some embodiments, the means of determining the target intervention path 406 may further include one or more of the following conditions: the shortest movement distance of the probe, the shortest intervention path, and the best imaging of the interventional object. A processor of an ultrasound imaging system may perform determination according to the above conditions to determine an optimal target intervention path.

In an embodiment of the present application, a target intervention starting point 416 may also be included. The target intervention starting point 416 may be an intersection point of the target intervention path 406 and an upper edge of the ultrasound image 401. As shown in FIG. 4, when the target intervention path 406 and the current intervention path 405 do not coincide (alternatively, it may be understood that the target intervention starting point 416 and the current intervention starting point 413 do not coincide), movement guidance related to the probe 402 may be generated and displayed on the ultrasound image. The movement guidance can guide the probe 402 to move such that the current intervention path 405 and the target intervention path 406 coincide.

The movement guidance may be a variety of types. For example, it could be an indication line 407 as shown in FIG. 4, the direction of the indication line 407 representing the direction in which the probe 402 moves. Optionally, the length of the indication line 407 may also represent the amount of translation required for the probe 402. Furthermore, both the target intervention path 406 and the current intervention path 405 themselves may also serve as movement guidance, and the user may move the probe himself by means of a difference between the two paths. In another example, the movement guidance may also be the current intervention starting point 413 and the target intervention starting point 416, and the difference between the two starting points can also serve the purpose of guiding the probe to move. In order to be able to disclose the technical solution of the present application more clearly, the indication line 407, the current intervention starting point 413/current intervention path 405, and the target intervention starting point 416/target intervention path 406 are simultaneously shown in FIG. 4. However, it will be understood that the different means described above can be freely combined; for example, only the target intervention starting point 416/target intervention path 406 is shown, or only the current intervention starting point 413/current intervention path 405 is shown, and the like, which will not be exhaustively listed.

Solutions of the present application provide a visual guide for the user to move the probe 402. With the assistance of the movement guidance, the user can thus move the probe 402 to a target position. As described above, the relative positions of the intervention starting point and the probe may be fixed. Although FIG. 4 does not show an intermediate procedure of translation of the probe 402, it is not difficult to understand that the current intervention starting point 413 may move in real time with movement of the probe 402, the relative positions of the current intervention starting point 413 and the probe 402 remaining unchanged during the movement. With such a configuration, the current intervention starting point 413 and the current intervention path 405 can always be determined, and thus can be compared with the target intervention starting point and the target intervention path. Due to the movement of the probe, the field of view of the ultrasound image will move with it, while the current intervention starting point 413 remains located at a fixed position in the field of view as it follows the movement of the probe.

Reference is made to FIG. 5. FIG. 5 is a schematic diagram 500 showing a probe guided to move after an intervention path is planned in some embodiments of the present application. After the user moves the probe 402 to the target position with the assistance of the probe movement guidance, the current intervention starting point 413 and the target intervention starting point 416 coincide, and the current intervention path 405 and the target intervention path 406 coincide. At this time, the current intervention path 405 avoids the non-intervention region 412. In FIG. 5, a dashed box portion 502 is a portion originally displayed in the ultrasound image 401, and in FIG. 5, the field-of-view range of the ultrasound image changes due to the movement of the probe 402, and an ultrasound image 501 does not include said portion, which is replaced with a new portion (a left portion of the image). In the solution of the present application, the movement of the probe 402 can ensure that the field of view of the ultrasound image moves with it, thereby ensuring, as much as possible, that the entire path is exposed within the field-of-view range of the ultrasound image 501.

In some embodiments of the present application, controlling an ultrasound beam transmitted by the probe so that the ultrasound beam is deflected in a direction perpendicular to the target intervention path is further included. Referring to FIG. 5, after the target intervention path 406 is determined, the processor may control the ultrasound beam (not shown) of the probe 402 to be deflected in a direction perpendicular to the target intervention path 406. In this way, when the current intervention path 405 and the target intervention path 406 coincide and a surgical intervention starts, an interventional object proceeds along the current intervention path 405, and the ultrasound beam can be as perpendicular as possible to the interventional object, thereby improving the imaging effect of the interventional object.

It should be understood that the timing of the described deflection may be after the target intervention path is determined and before an intervention operation. For example, the deflection may be performed once the target intervention path is determined. In this way, ultrasound imaging parameters during an intervention can be adjusted in advance as a backup, thereby increasing response speed of the system. The timing of the deflection may also be during movement of the probe or after movement ends.

Further, in some embodiments of the present application, improvements on the ultrasound imaging technique during an intervention are also performed. Specifically, some embodiments of the present application further include: identifying an interventional object in the ultrasound image; and generating and displaying an intervention process guide based on a positional relationship between the identified interventional object and the target intervention region. In this way, in conjunction with the probe movement guidance described above, more complete and comprehensive ultrasound imaging assistance can be provided to a doctor. For example, an interventional operation may be performed after the doctor has determined the position of the probe, the intervention starting points, and the intervention paths. During the interventional operation, by means of generating the intervention process guide, it is possible to better help a doctor to understand the process of the intervention, enhance the doctor's confidence, and improve operation efficiency.

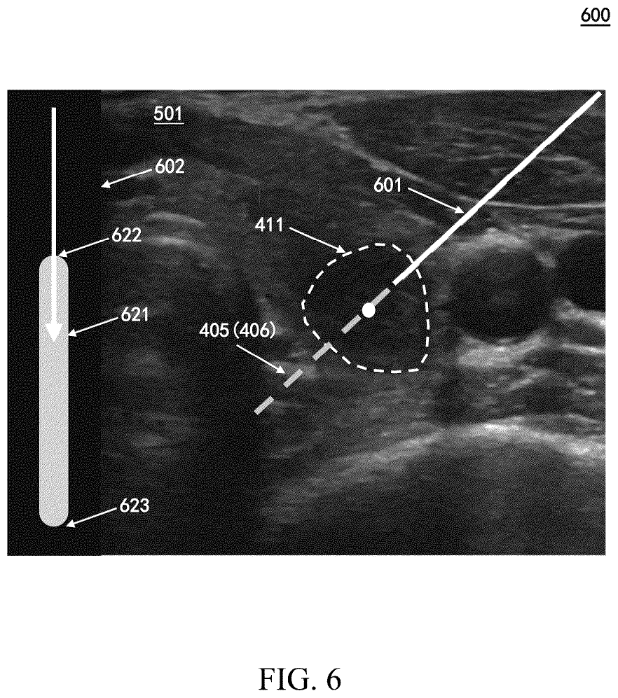

A further detailed description will be provided below with reference to the drawings. Reference is made to FIG. 6 which shows a schematic diagram 600 of ultrasound imaging during an intervention according to some embodiments of the present application. The ultrasound image 501 may be an ultrasound image generated after the probe moves and reaches the target position as described in the above embodiment. The current intervention path 405 and the target intervention path 406 coincide. In some embodiments, the current intervention path 405 may be overlaid on the ultrasound image 501 for display, to provide guidance for the doctor's interventional operation.

In addition, in order to better assist the interventional operation, an interventional object 601 in the ultrasound image 501 may also be identified in the present application. For specific identification means, reference may be made to the identification of an anatomical feature such as the target intervention region 411 and the non-intervention region in any of the embodiments described above, which will not be repeated here. Further, according to a positional relationship between an identified interventional object 601 and the target intervention region 411, an intervention process guide 602 may be generated and displayed. As shown in FIG. 6, the intervention process guide 602 may be configured to be displayed independently of the ultrasound image 501. In other words, in the example of the present application, the user can quickly know a current intervention process only by observing the intervention process guide 602, which is not easily interfered with by other factors such as the ultrasound image 501.

FIG. 6 shows a case in which the intervention process guide 602 and the ultrasound image 501 are displayed side by side, but it is understood that any other independent display means may be used within the teaching of the present disclosure. For example, being displayed floating on the ultrasound image 501.

Information displayed in the interventional process guide 602 may include, as shown: an endpoint 621 of the interventional object 601, a minimum distance 622 and a maximum distance 623 from the target intervention region 411 to the current interventional starting point 413. As described above, the information may be acquired by identifying the endpoint 621, the intersection point of the current intervention path 405 and the upper edge of the target intervention region 411 (i.e., the minimum distance 622), and the intersection point of the current intervention path 405 and a lower edge of the target intervention region 411 (i.e., the maximum distance 623) using an image recognition technique. It will be appreciated that the endpoint 621, the minimum distance 622, and the maximum distance 623 already include depth information needed for the intervention. That is, the endpoint 621 should reach between the minimum distance 622 and the maximum distance 623. In this way, the user can intuitively and quickly understand the process of the surgical intervention.

In an alternative example, the intervention process guide 602 does not provide information of an intervention angle of the interventional object 601. In such a configuration, an effect of guidance on an intervention depth of the interventional process guide 602 is more compact, redundant information display is eliminated as much as possible, and unnecessary interference with the user is avoided. Also, since the intervention paths have already been planned in advance in the example of the present application, the intervention process guide 602 does not provide angle information, nor does it cause a lack of this dimension of information.

In an alternative example, as shown in FIG. 6, the endpoint 621 of the interventional object, the minimum distance 622 and the maximum distance 623 are displayed in a same linear direction. Such a display means facilitates the user more intuitively understanding the depth information of the endpoint 621 of the interventional object compared to the target intervention region, thereby further improving working efficiency.

To this end, any of the above embodiments of the present application provide a quick and accurate assisted imaging means for a doctor before and/or during a surgical intervention, which facilitates improvement of the doctor's work efficiency and accuracy during the operation. Any of the above embodiments may be freely combined under the teachings of the present application, unless otherwise specified.

Some embodiments of the present application further provide an ultrasound imaging system, which may be as shown in FIG. 1 or any other one. The system includes: a probe, comprising an ultrasound transducer; a processor, configured to execute the method described in any of the above embodiments; and a display apparatus, used to receive a signal from the processor and perform a display operation.

Some embodiments of the present application further provide a non-transitory computer-readable medium, where the non-transitory computer-readable medium has a computer program stored thereon, the computer program has at least one code segment, and the at least one code segment is executable by a machine so as to enable the machine to execute the steps of the method in any of the above embodiments.

Correspondingly, the present disclosure may be implemented by means of hardware, software, or a combination of hardware and software. The present disclosure may be implemented in at least one computer system in a centralized manner, or implemented in a distributed manner; and in the distributed manner, different elements are distributed on a plurality of interconnected computer systems. Any type of computer system or other apparatus suitable for implementing the methods described herein is considered to be appropriate.

Various embodiments may also be embedded in a computer program product, which includes all features capable of implementing the methods described herein, and the computer program product is capable of executing these methods when loaded into a computer system. The computer program in this context means any expression in any language, code, or symbol of an instruction set intended to enable a system having information processing capabilities to execute a specific function directly or after any or both of the following: a) conversion to another language, code, or symbol; and b) replication in different material forms.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, and changes can also be made to the present invention and should be included in the scope of protection of the present invention as long as these changes do not depart from the spirit of the present invention.

1. A method for guiding movement of a probe, so as to perform ultrasound imaging, comprising:
using the probe to acquire a real-time ultrasound image related to a tissue to be imaged;
determining a current intervention starting point, and determining a target intervention region and a non-intervention region in the ultrasound image;
determining a current intervention path based on the target intervention region and the current intervention starting point, and determining a target intervention path based on the target intervention region and the non-intervention region; and
based on the current intervention path and the target intervention path, generating and displaying movement guidance related to the probe, the movement guidance being configured to guide the probe to move such that the current intervention path and the target intervention path coincide.

2. The method according to claim 1, wherein the current intervention starting point is configured to be close to a side edge of the probe and located at an upper edge of the ultrasound image.

3. The method according to claim 1, wherein the current intervention starting point moves in real time with the movement of the probe, and during said movement, the relative positions of the current intervention starting point and the probe remain unchanged.

4. The method according to claim 1, further comprising:
displaying the current intervention path and the target intervention path in real time during the movement of the probe.

5. The method according to claim 1, wherein the current intervention path is a line connecting a point in the target intervention region to the current intervention starting point.

6. The method according to claim 1, wherein the target intervention path passes through a point of the target intervention region and does not pass through the non-intervention region.

7. The method according to claim 1, further comprising:
simultaneously displaying a target intervention starting point and the current intervention starting point in real time during the movement of the probe; wherein the target intervention starting point is an intersection point of the target intervention path and the upper edge of the ultrasound image; and/or
simultaneously displaying the current intervention path and the target intervention path in real time during the movement of the probe.

8. The method according to claim 1, further comprising:
controlling an ultrasound beam transmitted by the probe, causing the ultrasound beam to deflect in a direction perpendicular to the target intervention path.

9. The method according to claim 1, further comprising:
identifying an interventional object in the ultrasound image; and
generating and displaying an intervention process guide based on a positional relationship between the identified interventional object and the target intervention region.

10. The method according to claim 9, wherein:
information displayed in the intervention process guide comprises: an endpoint of the interventional object, and a minimum distance and a maximum distance from the target intervention region to the current intervention starting point; and
the intervention process guide is displayed independently of the ultrasound image.

11. The method according to claim 10, wherein the interventional process guide does not provide information of an intervention angle of the interventional object.

12. The method according to claim 10, wherein the endpoint of the interventional object, the minimum distance, and the maximum distance are displayed in a same linear direction.

13. An ultrasound imaging system, comprising:
a probe, the probe transmitting an ultrasound beam to tissue to be imaged, and receiving an echo signal;
a processor configured to:
acquire a real-time ultrasound image obtained by the probe that is related to a tissue to be imaged;
determine a current intervention starting point, and determining a target intervention region and a non-intervention region in the ultrasound image;
determine a current intervention path based on the target intervention region and the current intervention starting point, and determining a target intervention path based on the target intervention region and the non-intervention region; and
based pm the current intervention path and the target intervention path, generate and displaying movement guidance related to the probe, the movement guidance being configured to guide the probe to move such that the current intervention path and the target intervention path coincide; and
a display apparatus, the display apparatus receiving a control of the processor and performing a display operation.

14. A non-transitory computer-readable medium, the non-transitory computer-readable medium having a computer program stored therein, the computer program having at least one code segment, and the at least one code segment being executable by a machine, to cause the machine to execute the steps of:
using the probe to acquire a real-time ultrasound image related to a tissue to be imaged;
determining a current intervention starting point, and determining a target intervention region and a non-intervention region in the ultrasound image;
determining a current intervention path based on the target intervention region and the current intervention starting point, and determining a target intervention path based on the target intervention region and the non-intervention region; and
based on the current intervention path and the target intervention path, generating and displaying movement guidance related to the probe, the movement guidance being configured to guide the probe to move such that the current intervention path and the target intervention path coincide.

15. The non-transitory computer readable medium of claim 14, wherein the current intervention starting point is configured to be close to a side edge of the probe and located at an upper edge of the ultrasound image.

16. The method according to non-transitory computer readable medium of claim 1, wherein the at least one code segment cause the machine to execute the further steps of, wherein the current intervention starting point moves in real time with the movement of the probe, and during said movement, the relative positions of the current intervention starting point and the probe remain unchanged.

17. The non-transitory computer readable medium of claim 14, wherein the at least one code segment cause the machine to execute the further step of:
displaying the current intervention path and the target intervention path in real time during the movement of the probe.

18. The non-transitory computer readable medium of claim 14, wherein the current intervention path is a line connecting a point in the target intervention region to the current intervention starting point.

19. The non-transitory computer readable medium of claim 14, wherein the target intervention path passes through a point of the target intervention region and does not pass through the non-intervention region.

20. The non-transitory computer readable medium of claim 14, wherein the at least one code segment cause the machine to execute the further steps of:
simultaneously displaying a target intervention starting point and the current intervention starting point in real time during the movement of the probe; wherein the target intervention starting point is an intersection point of the target intervention path and the upper edge of the ultrasound image; and/or
simultaneously displaying the current intervention path and the target intervention path in real time during the movement of the probe.
